(19) United States
(12) Patent Application Publication
(10) Pub. No.: US 2010/0131060 A1
(43) Pub. Date: May 27, 2010

(54) Diffractive multifocal intraocular lens with modified central distance zone

(76) Inventors: Michael J. Simpson, Arlington, TX (US); Krishnakumar Venkateswaran, Burleson, TX (US)

Correspondence Address: ALCON, IP LEGAL, TB4-8, 6201 SOUTH FREEWAY, FORT WORTH, TX, 76134, US

(21) Appl. No.: 12/621,613

(22) Filed: Nov. 19, 2009

Related U.S. Application Data
(60) Provisional application No. 61/116,458, filed on Nov. 20, 2008.

(51) Int. Cl. A61F 2/16

(52) U.S. Cl. 623/6.24

(57) ABSTRACT

The present invention generally provides multifocal ophthalmic lenses, e.g., multifocal intraocular lenses, that employ a central refractive region for providing a refractive focusing power and a diffractive region for providing diffractive focusing powers. The refractive focusing power provided by the lens's central region corresponds to a far-focusing power that is substantially equal to one of the diffractive focusing powers while the other diffractive power corresponds to a near-focusing power. The far-focusing power can be enhanced by changes to the phase of the central refractive region and/or changes to the curvature of the central refractive region.

This application claims priority to U.S. Provisional Application Ser. No. 61/116,458 filed on Nov. 20, 2008.

### BACKGROUND

The present invention relates generally to multifocal ophthalmic lenses, and, more particularly, to multifocal intraocular lenses that can provide refractive and diffractive optical focusing powers.

Intraocular lenses (IOLs) are routinely implanted in patients' eyes during cataract surgery to replace a natural crystalline lens. Some IOLs employ diffractive structures to provide a patient with not only a far-focus power but also a near-focus power. In other words, such IOLs provide the patient with a degree of accommodation (sometimes referred to as “pseudoaccommodation”). The division of energy between the far-focus and the near-focus lens powers can be adjusted by modifying the “step heights” of the diffractive structure, and by the use of a central “refractive” zone that directs light solely to a single focus. An increase of energy to one focus generally causes a reduction of energy to the other focus, which reduces image contrast for that focus. However, image contrast is also affected by other factors, such as imaging aberrations, and the characteristics of the diffractive structure.

Accordingly, there is a need for diffractive multifocal lens designs that will enhance image contrast for both the far-focus and the near-focus.

### SUMMARY

In one aspect, the present invention provides an intraocular lens (IOL), which comprises an optic having an anterior surface and a posterior surface, where the optic includes a central refractive region for providing a refractive focusing power. A diffractive region is disposed on at least one of the lens surfaces for providing a near and far diffractive focusing power. In some cases, the refractive and diffractive far focusing powers are substantially equal. The optical properties of light passing through the central zone can be adjusted to optimize overall image contrast for both powers.

In a related aspect, in the above IOL, one of the surfaces (e.g., the anterior surface) includes a central refractive region that is surrounded by a diffractive region, which is in turn surrounded by an outer refractive region. In some cases, the central refractive region can have a diameter in a range of about 0.5 mm to about 2 mm.

In another aspect, the diffractive region includes a plurality of diffractive zones (e.g., 2 to 20 zones) that are separated from one another by a plurality of steps. The height of the central step, and/or the curvature of the central zone, are adjusted to optimize image contrast. While in some cases the other steps exhibit substantially uniform heights, in others their heights can be non-uniform. For example, the steps can be apodized such that their heights decrease as a function of increasing radial distance from a center of the optic. Alternatively, the apodized steps can exhibit increasing heights as a function of increasing radial distance from the center of the optic—that is the steps can be “reverse apodized.” In another case, the step heights can increase from an inner radial boundary of the diffractive region to an intermediate location in that region followed by a decrease to the region's outer radial boundary, and vice versa.

In another aspect, a multifocal ophthalmic lens (e.g., an IOL) is disclosed, which includes an optic having an anterior surface and a posterior surface configured such that the optic includes a central refractive and an outer refractive region. In addition, a diffractive region is disposed on at least one of the surfaces to provide two diffractive focusing powers.

In some cases, in the above ophthalmic lens, the central and the outer refractive regions provide different refractive powers, e.g., the central region can provide a far-focusing power and the outer refractive region can provide a near-focusing power. The diffractive region can, in turn, provide diffractive near and far focusing powers corresponding to the refractive near and far focusing powers provided by the central and the outer regions.

In each of these aspects, the central refractive region of the embodiments of the ophthalmic lens of the present invention comprises a central distance zone, and the diffractive region can have reduced step heights, both cooperating to increase the amount of energy directed to the distance power of the IOL optic while maintaining an acceptable level of near-focusing power.

Further understanding of various aspects of the invention can be obtained by reference to the following detailed description in conjunction with the associated drawings, which are discussed briefly below.

### BRIEF DESCRIPTION OF THE DRAWINGS

FIG. 1 is a schematic side view of a prior art apodized diffractive multifocal IOL;

FIG. 2A is a schematic top view of a multifocal IOL according to one embodiment of the invention;

FIG. 2B is a schematic side view of a the multifocal IOL shown in FIG. 2A having a central distance zone with an adjusted central zone phase and approximately the same curvature as the base curve;

FIG. 2C shows a radial profile of the anterior surface of the IOL shown in FIGS. 2A and 2B from which the base profile of the anterior surface has been subtracted;

FIG. 3 is a schematic side view of a multifocal IOL in accordance with one embodiment of the present invention having a central distance zone with an adjusted central zone phase and central zone slope;

FIG. 4 is a series of graphs illustrating the optical properties as a function of off-axis distance squared of embodiments of the present invention having different central zone phase and central zone curvature combinations;

FIG. 5 is a series of graphs illustrating changes in the Modulation Transfer Function for embodiments of an IOL of the present invention having different central phase values;

FIG. 6A is a schematic side view of a multifocal IOL in accordance with one embodiment having a reverse-apodized diffractive region;

FIG. 6B is a radial profile of the anterior surface (minus the base profile of the surface) of the IOL shown in FIG. 6A;

FIG. 6C is a schematic side view of a multifocal IOL according to an embodiment of the invention;

FIG. 6D is a radial profile of the anterior surface (minus the surface base profile) of the IOL of FIG. 6C, indicating that the steps separating different diffractive zones of a diffractive region disposed on the surface exhibit an increase in heights followed by a decrease as a function of increasing radial distance from the lens center;

FIG. 6E is a radial profile of a surface (minus the surface base profile) of an IOL according to an embodiment in which the steps separating different diffractive zones of a diffractive region disposed on the surface exhibit a decrease in heights followed by an increase as a function of increasing radial distance from the lens center;

FIG. 7 is a radial profile of a surface (minus the surface base profile) of an IOL according to an embodiment in which the steps separating different diffractive zones of a diffractive region disposed on the surface exhibit substantially uniform heights;

FIG. 8 is a schematic side view of an IOL according to an embodiment of the invention in which a diffractive region disposed on the lens's anterior surface extends to the periphery of the lens; and

FIG. 9 is a schematic side view of an IOL according to an embodiment of the invention having a central refractive region and an outer refractive region, which provide different refractive focusing powers.

### DETAILED DESCRIPTION

The present invention generally provides multifocal ophthalmic lenses, e.g., multifocal intraocular lenses, that employ a refractive region for providing a refractive focusing power and a diffractive region for providing one or more diffractive focusing powers. In some cases the refractive focusing power provided by the lens corresponds to a far-focus optical power that is substantially equal to one of the diffractive focusing powers while the other diffractive power corresponds to a near-focus optical power. As such, in some cases, the focusing properties of the lenses are dominated by their far-focus ability, especially for small pupil sizes. In the embodiments that follow, the salient features of various aspects of the invention are discussed in connection with intraocular lenses (IOLs). The teachings of the invention can also be applied to other ophthalmic lenses, such as contact lenses. The term “intraocular lens” and its abbreviation “IOL” are used herein interchangeably to describe lenses that are implanted into the interior of the eye to either replace the eye's natural lens or to otherwise augment vision regardless of whether or not the natural lens is removed. Intracorneal lenses and phakic intraocular lenses are examples of lenses that may be implanted into the eye without removal of the natural lens.

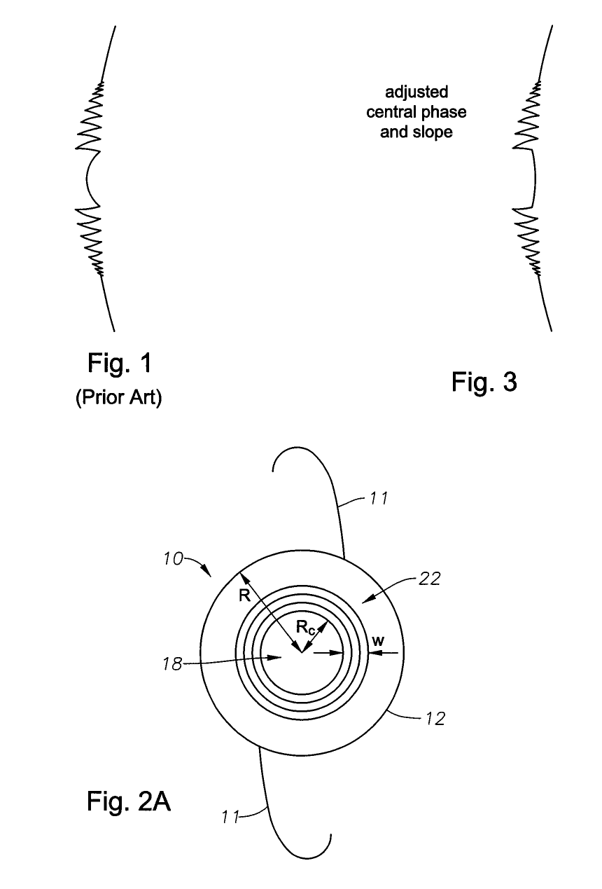

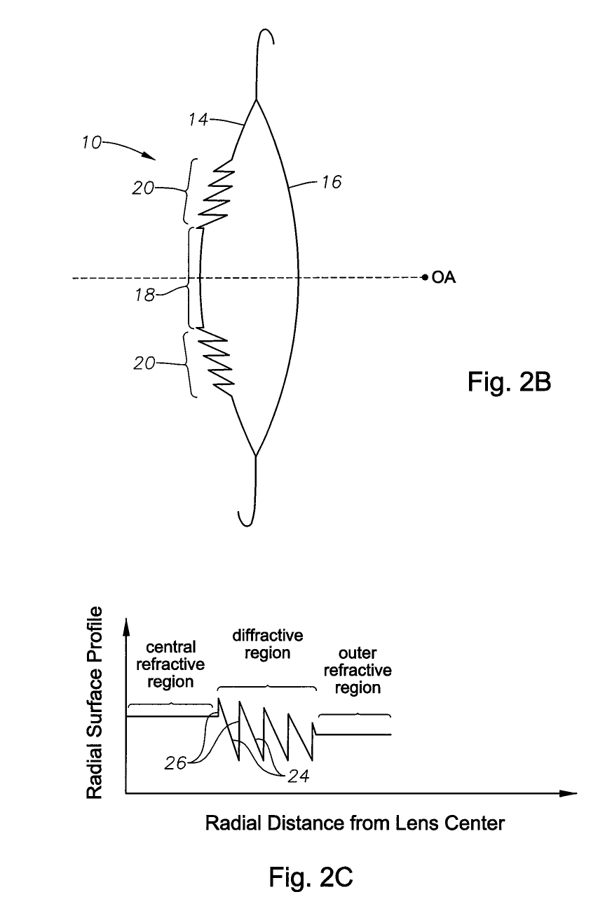

FIG. 1 schematically depicts a prior art apodized diffractive multifocal IOL lens surface, where the curvature of the central zone is broadly similar to the curvature of the adjacent annular zone. FIGS. 2A, 2B and 2C schematically depict a multifocal intraocular lens (IOL) 10 according to one embodiment of the present invention that includes an optic 12 having an anterior surface 14 and a posterior surface 16, which are disposed about an optical axis OA. As discussed in more detail below, the IOL 10 provides a far as well as a near focusing power. While in this embodiment, the IOL has a bi-convex profile (each of the anterior and posterior surfaces has a convex profile), in other embodiments, the IOL can have any other suitable profile, e.g., convex-concave, piano-convex, etc. In some implementations, the optic 12 can have a maximum radius (R) from the optical axis OA in a range of about 2 mm to about 4 mm, while in other embodiments it can be larger. To direct more light to the distance focus, for example, all the step heights of the diffractive steps are reduced compared to the prior art example in FIG. 1. This has the effect of directing more light to the distance focus and less light to the near focus.

In addition to changes in the diffractive step heights, the anterior surface 14 includes a central “refractive” region 18, which is surrounded by an annular diffractive region 20, and an outer refractive region 22. If the central region has a “refractive” focus that corresponds to the far power of the lens, then additional light is directed to that lens power. In many implementations, the central refractive region 18 can have a radius (R_{c}) relative to the optical axis OA in a range of about 0.25 mm to about 1 mm—though other radii can also be employed. In this exemplary embodiment, the posterior surface 16 does not include any diffractive structures, though in other embodiments it can include such structures. As discussed further below, the central refractive region 18 of the anterior surface contributes to the focusing power of the optic, which corresponds in this embodiment to the IOL's far-focus optical power. By way of example, in some cases, the optic's distance power can be in a range of about −5 to about +55 Diopters and more typically in a range of about 6 to about 34 Diopters, or in a range of about 18 to about 26 Diopters.

In the example of FIGS. 2A-2C, the base profiles of both the anterior surface 14 and the posterior surface 16 are substantially spherical with curvatures that are chosen, together with the index of refraction of the material forming the optic to provide a lens with the distance power only, in the absence of the diffractive structure. However, the axial location of the central zone region is adjusted so that it does not match the base curve. This is also indicated by the height separation between the central and outer regions in FIG. 2C. This adjustment of the relative optical phase of the central zone, compared to the rest of the lens, can be used to adjust image contrast for both lens powers somewhat independently from the division of energy to the two foci. Similarly, the curvature of the surface of the central zone can also be adjusted, either alone or in conjunction with a phase delay at the diffractive step, in order to optimize the image contrast.

In some other implementations, one or both lens surfaces can exhibit aspherical base profiles adapted to control aberrations and increase image contrast. By way of example, an IOL in accordance with such an embodiment can comprise an optic having an anterior surface and a posterior surface. The anterior surface can include a refractive central region that generates, in cooperation with the posterior surface, a refractive optical power. Similar to the previous embodiment, a diffractive region can surround the refractive central region. The diffractive region can, in turn, be surrounded by a refractive outer region. In such an embodiment, the anterior surface has an aspheric base profile. In other words, the base profile of the anterior surface differs from a putative spherical profile. For example, the aspheric base profile of the anterior surface can be characterized by a negative conic constant, which can be selected based on the refractive power of the lens, that controls aberration effects. By way of example, the conic constant can be in a range of about −10 to about −1000 (e.g., −27). Though in this embodiment, the base profile of the posterior surface is substantially spherical, in other embodiments, the base profile of the posterior surface can also exhibit a selected degree of asphericity such that the combined aspherical profiles of the two surfaces would facilitate the generation of a single refractive focus by the central portion of the lens. In other implementations, the central refractive zone can have a spherical profile in order to facilitate the generation of a single refractive focus, even when the surface has an otherwise aspheric base profile.

Referring again to FIGS. 2A, 2B and 2C, the optic 12 can be formed of any suitable biocompatible material. Some examples of such materials include, without limitation, soft acrylic, silicone, hydrogel or other biocompatible polymeric materials having a requisite index of refraction for a particular application of the lens. In many implementations, the index of refraction of the material forming the optic can be in a range of about 1.4 to about 1.6 (e.g., the optic can be formed of a lens material commonly known as Acrysof® (a cross-linked copolymer of 2-phenylethyl acrylate and 2-phenylethyl methacrylate) having an index of refraction of 1.55)

The exemplary IOL 10 also includes a plurality of fixation members (e.g., haptics) 11 that facilitate placement of the IOL in a patient's eye. The fixation members 11 can also be formed of suitable polymeric materials, such as polymethylmethacrylate, polypropylene and the like.

As noted above, the optic 12 also includes a diffractive region 20, which is disposed on its anterior surface 14, though in other embodiments it can be disposed on the posterior surface or on both surfaces. The diffractive region 20 forms an annular region surrounding the central refractive region 18 of the optic's anterior surface. In this exemplary embodiment, the diffractive region 20 provides a far-focus optical power as well as a near-focus power. In this example, the far-focus optical power provided by the diffractive structure is substantially similar to the refractive focusing power provided by the IOL's central refractive region. The near-focus optical power provided by the diffractive region can be, e.g., in a range of about 1 D to about 4 D, though other values can also be used. In some implementations, the diffractive region 20 can have a width (w) in a range of about 0.5 mm to about 2 mm, though other values can also be employed. In other embodiments the diffractive region 20 can provide a far-focus optical power and not a near-focus power.

Although in some embodiments the diffractive region can extend to the outer boundary of the optic 12, in this embodiment, the diffractive region is truncated. More specifically, the diffractive region is disposed between the lens's central refractive region 18 and its outer refractive region 22. Similar to the refractive central region, the outer refractive region provides a single refractive focusing power, which in this case is substantially equal to the refractive power provided by the central region. In other words, the IOL's central and the outer refractive regions contribute only to the lens's far-focus power, while the diffractive region (herein also referred to as the zonal diffractive region) directs light energy incident thereon into both the far and near foci of the lens. As will be described herein, the energy directed to the far-focus power can be increased by reducing the diffractive region step heights and/or by adjusting the curvature of the central refractive distance zone.

As shown schematically in FIG. 2C, which is a surface profile of the anterior surface without the base profile of the surface, in this exemplary embodiment, the diffractive region 20 is formed of a plurality of diffractive zones 24 disposed on an underlying base curve of the anterior surface 14. The number of the diffractive zones can be in a range of about 2 to about 20, though other numbers can also be employed. The diffractive zones 24 are separated from one another by a plurality of steps 26. In this exemplary implementation, the heights of the steps 26 are non-uniform. More specifically, in this example, the step heights decrease as a function of increasing distance from a center of the anterior surface (the intersection of the optical axis OA with the anterior surface). In other words, the steps are apodized to exhibit decreasing heights as a function of increasing radial distance from the lens's optical axis. As discussed in more detail below, in other embodiments, the step heights can exhibit other types of non-uniformity, or alternatively, they can be uniform. The schematic radial profile depicted in FIG. 2C also shows that the curvatures of the IOL's central and outer refractive regions correspond to the base curvature of the anterior surface (hence these regions are shown as flat in the figure), though a phase shift is given to the central zone. Other configurations, as described below, can also be used to divert more energy to the far-focus power of the embodiments of the present invention.

The steps are positioned at the radial boundaries of the diffractive zones. In this exemplary embodiment, the radial location of a zone boundary can be determined in accordance with the following relation:

rᵢ²=r₀²+2i∂∫ Equation (1),

wherein

i denotes the zone number

r₀ denotes the radius of the central refractive zone,

∂ denotes the design wavelength, and

∫ denotes a focal length of the near focus.

In some embodiments, the design wavelength ∂ is chosen to be 550 nm green light at the center of the visual response. In some cases, the radius of the central zone (r₀) can be set to be \sqrt{∂∫}.

With continued reference to FIG. 2C, in some cases, the step height between adjacent zones, or the vertical height of each diffractive element at a zone boundary, can be defined according to the following relation:

$$Step Height = \frac{\partial }{2 ( {n}_{2} - {n}_{1} )} fapodize , \qquad Equation ( 2 )$$

wherein

∂ denotes the design wavelength (e.g., 550 nm),

n₂ denotes the refractive index of the material from which the lens is formed,

n₁ denotes the refractive index of a medium in which the lens is placed,

and fapodize represents a scaling function whose value decreases as a function of increasing radial distance from the intersection of the optical axis with the anterior surface of the lens. For example, the scaling function can be defined by the following relation:

$$fapodize = 1 - {\{ \frac{( {r}_{i} - {r}_{in} )}{( {r}_{out} - {r}_{in} )} \}}^{exp} , {r}_{in} \leq {r}_{i} \leq {r}_{out} , \qquad Equation ( 3 )$$

wherein

rᵢ denotes the radial distance of the i^{th} zone,

rᵢₙ denotes the inner boundary of the diffractive region as depicted schematically in FIG. 2C,

rₒᵤₜ denotes the outer boundary of the diffractive region as depicted schematically in FIG. 2C, and

exp is a value chosen based on the relative location of the apodization zone and a desired reduction in diffractive element step height. The exponent exp can be selected based on a desired degree of change in diffraction efficiency across the lens surface. For example, exp can take values in a range of about 2 to about 6.

As another example, the scaling function can be defined by the following relation:

$$fapodize = 1 - {( \frac{{r}_{i}}{{r}_{out}} )}^{3} , \qquad Equation ( 4 )$$

wherein

rᵢ denotes the radial distance of the i^{th} zone, and

rₒᵤₜ denotes the radius of the apodization zone.

Referring again to FIG. 2C, in this exemplary embodiment, each step at a zone boundary is centered about the base profile with half of its height above the base profile and the other half below the profile, apart from the central step. Further details regarding selection of the step heights, other than the height of the central step, can be found in U.S. Pat. No. 5,699,142 which is herein incorporated by reference in its entirety.

In use, the central refractive region provides a single far focus refractive power such that the IOL 10 effectively functions as a monofocal refractive lens for small pupil sizes, that is the pupil sizes less than or equal to the radial size of the central refractive region. For larger pupil sizes, while the central region continues to provide a single far-focus optical power, the diffractive region begins to contribute to the IOL's focusing power by providing two diffractive focusing powers: one substantially equal to the refractive far-focus power of the central region and the other corresponding to a near-focus power. As the pupil size increases further, the outer refractive region 22 can also contribute—refractively—to the far-focus power of the lens. The fraction of the light energy distributed to the near focus relative to the far focus can be adjusted, e.g., via the sizes of the central and outer refractive regions as well as the parameters (e.g., step heights) associated with the diffractive region. Further, in cases in which the step heights are apodized, this fraction can change as a function of the pupil size. For example, the decrease in the step heights of the diffractive structure results in an increase in the fraction of the light energy transmitted to the far focus by the diffractive structure as the pupil size increases.

The energy directed to the far-focus power of the embodiments of the present invention can thus be increased by reducing the diffractive step heights in the diffractive region and/or by adjusting the central distance zone curvature. The central zone could have the same curvature as the base curvature of the IOL, resulting in a simple central distance zone, or could be enhanced for improved far-focus performance by having a different central zone curvature. For example, FIG. 2B shows a schematic side view of the multifocal IOL of FIG. 2A having a central distance zone with an adjusted central zone phase and approximately the same curvature as the base curve. In this embodiment, the central zone phase is adjusted by adjusting (reducing) the height of the first diffractive step (the step closest to the central zone) and thereby changing the phase delay of the central zone. In another embodiment, such as shown in FIG. 3, a multifocal IOL in accordance with the present invention is depicted having a central distance zone with an adjusted central zone phase and a central zone curvature adjusted to differ from that of the IOL base curve to control the image quality for both lens powers.

As can be seen from FIGS. 2B and 3, and other FIGUREs herein, different lens parameters can be used alone or in combination to increase the energy distributed to the far-focus power of the embodiments of the present invention, and to control the image contrast. A central diffractive step adjustment can thus be combined with a change in central zone curvature, for example. The shape of the central zone can also be adjusted, and can be spherical or aspheric and differ from that of the base curve. Further, the adjustments to the central zone phase and curvature described herein can likewise be used to increase the energy distributed to the near-focus power in some embodiments as opposed to the far-focus power. Thus, the embodiments of the present invention can quite effectively be used to direct more energy to a first (far-focus) lens power or to a second (near-focus) lens power, while controlling image quality.

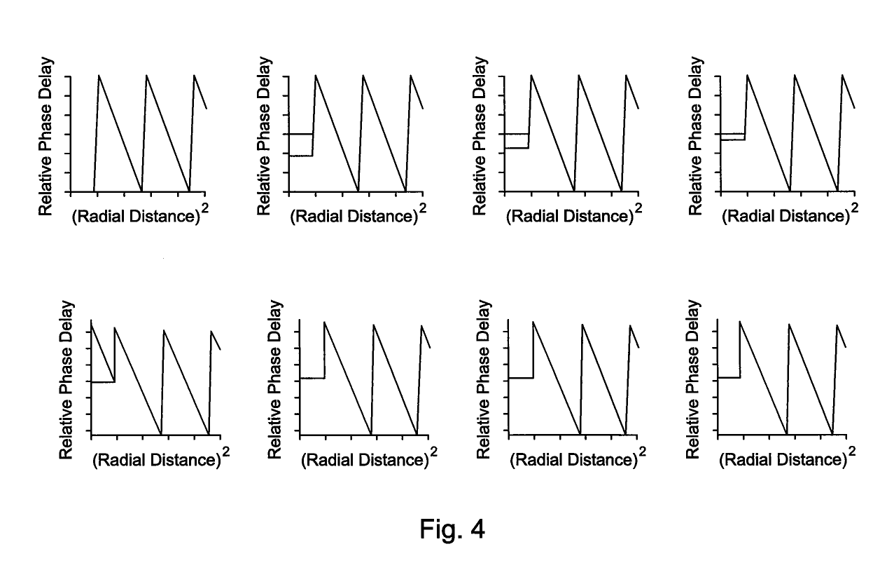

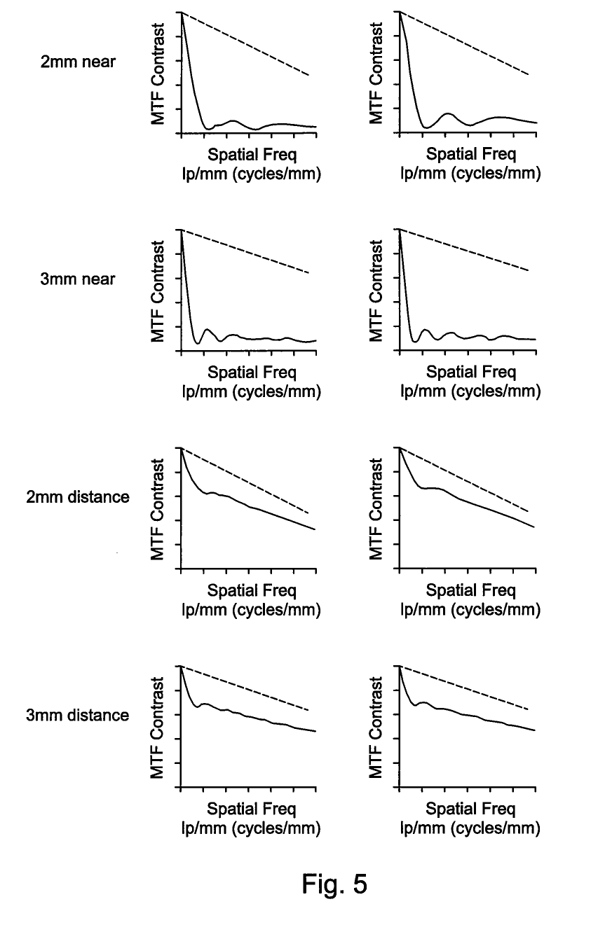

FIG. 4 is a series of graphs illustrating the optical properties as a function of off-axis distance squared of embodiments of the present invention having different central zone phase and central zone curvature combinations. FIG. 5 is a series of graphs illustrating changes in the Modulation Transfer Function (MTF) for embodiments of an IOL of the present invention having different central phase values. These graphs illustrate examples of adjustments to the central zone when plotted as a function of the square of the IOL radius. They represent the optical phase delay at the surface of the central zone and also the physical surface profile of the IOL optic. FIG. 5 illustrates the improvements in far-focus power by a specific example of a prior art lens design having a simple central distance zone compared to an embodiment of an IOL of the present invention having a phase delay of 0.5 at the central diffractive step. In addition, this example shows increased energy to the far-focus power as compared to the prior art lens by reducing all of the diffractive region step heights. In this example, the MTF contrast increases for the near power with the introduction of the central zone phase delay, compared to a similar lens where there is no phase delay for the central zone.

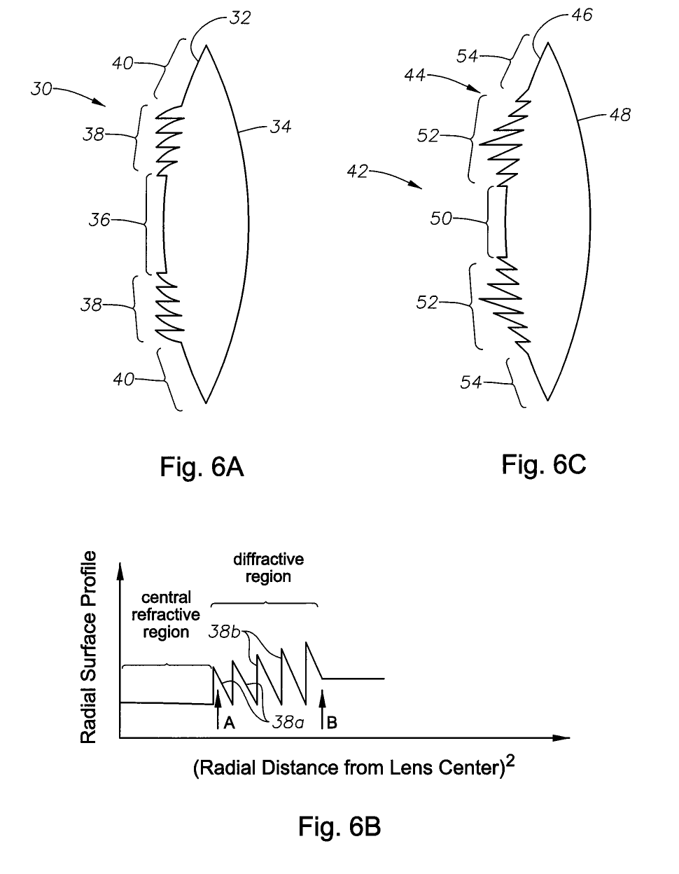

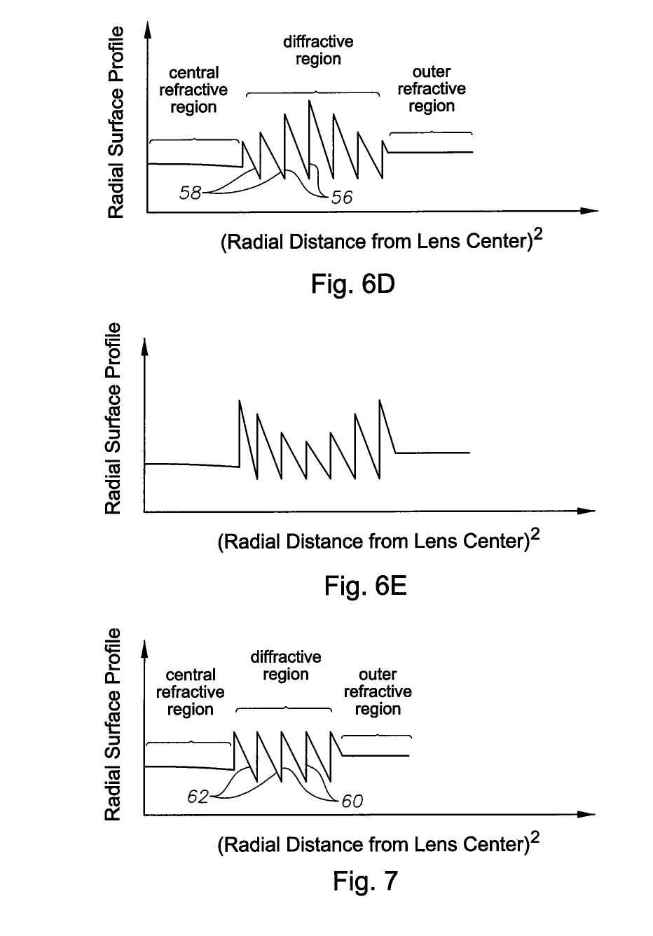

The apodization of the diffractive region of the embodiments of this invention is not limited to the one discussed above. In fact, a variety of types of apodization of the step heights can be employed. By way of example, with reference to FIGS. 6A and 6B, in some embodiments, an IOL 30 can include an anterior surface 32 and a posterior surface 34, where the anterior surface is characterized by a central refractive region 36, an annular diffractive region 38 that surrounds the central refractive region 34, and an outer refractive region 40. The annular diffractive region is formed by a plurality of diffractive zones 38a that are separated from one another by a plurality of steps 38b, where the steps exhibit increasing heights from an inner boundary A of the diffractive region to an outer boundary B thereof.

Such an apodization of the step heights is herein referred to as “reverse apodization.” Similar to the previous embodiment, the diffractive region contributes not only to the IOL's far-focus optical power but also to its near-focus power, e.g., the near-focus power can be in a range of about 1 to about 4 D. However, unlike the previous embodiment, the percentage of the incident light energy transmitted by the diffractive region to the far focus decreases as the pupil size increases (due to the increase in the step heights as a function of increasing radial distance from the optical axis).

In other embodiments, the step heights in the diffractive region can increase from the region's inner boundary to reach a maximum value at an intermediate location within that region followed by a decrease to the region's outer boundary. By way of example, FIG. 6C depicts such an IOL 42 having an optic 44 characterized by an anterior surface 46 and a posterior surface 48. Similar to the previous embodiments, the anterior surface 46 includes a central refractive region 50, an annular diffractive region 52 that surrounds the refractive region, and an outer refractive region 54 that in turn surrounds the diffractive region. With reference to the radial profile of the anterior surface presented in FIG. 6D, the annular diffractive region includes a plurality of diffractive zones 56 separated from another by a plurality of steps 58, where the step heights exhibit an increase followed by a decrease as a function of increasing radial distance from the center of the lens. Alternatively, in another embodiment shown schematically in FIG. 6E, the step heights show a decrease followed by an increase as a function of increasing distance from the lens center.

In yet other embodiments, the step heights separating different zones of the diffractive region can be substantially uniform (e.g., within manufacturing tolerances). By way of illustration, FIG. 7 schematically depicts a radial profile of a surface of such a lens (e.g., the anterior surface of the lens) from which the underlying base profile has been subtracted. The radial surface profile indicates that the surface includes a central refractive region A (with a curvature that is substantially equal to the base curvature of the surface, but with an additional phase delay), a diffractive region B and an outer refractive region C. The diffractive region B is characterized by a plurality of diffractive zones 60 that are separated from one another by a plurality of steps 62. The heights of the steps 62 are substantially uniform.

By way of example, in some implementations of an IOL having a substantially uniform step height, which provides a selected phase shift at each zone boundary, the radial location of a zone boundary can be determined in accordance with equation 1. In some cases, the radius of the central zone (r₀) can be set to be \sqrt{∂∫}. Further, the step height between adjacent zones can be defined in accordance with the following relation:

$$Step Height = \frac{b \partial }{( {n}_{2} - {n}_{1} )} \qquad Equation ( 5 )$$

wherein

∂ denotes the design wavelength (e.g., 550 nm),

n₂ denotes the refractive index of the material from which the lens is formed,

n₁ denotes the refractive index of the medium in which the lens is placed, and

b is a fraction, e.g., 0.5 or 0.7.

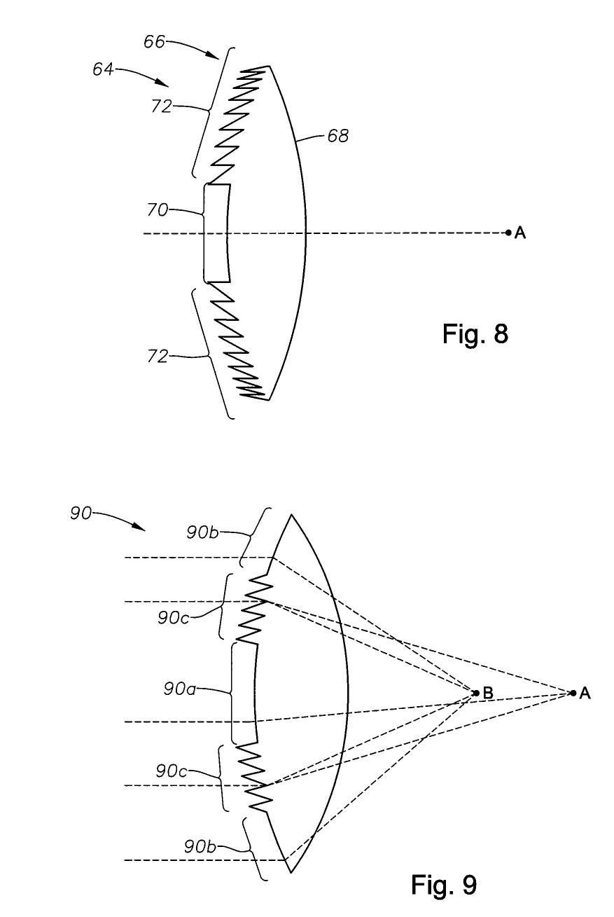

In some embodiments, the diffractive region can extend from the outer boundary of the central refractive region to the outer boundary of the optic. By way of example, FIG. 8 schematically depicts such an IOL 64 that includes an anterior surface 66 and a posterior surface 68. The anterior surface includes a central refractive region 70 that, in cooperation with the refractive posterior surface, imparts to the optic a refractive far-focus power. The central zone has an adjustment in step height and/or curvature. A diffractive region 72 disposed on the anterior surface extends from the outer boundary of the central refractive region to the outer boundary of the optic, and provides a diffractive near-focus and a diffractive far-focus optical power. In this exemplary implementation, the diffractive far-focus power is substantially equal to the refractive far-focus power provided by the optic's refractive central region. Although in this example the diffractive region is formed by a plurality of diffractive zones separated by steps having substantially uniform heights, in other implementations the step heights can be non-uniform (e.g., they can be apodized).

In some other embodiments, an IOL can include a central refractive region, an annular diffractive region disposed on a surface thereof, and an outer refractive region, where the central and the outer refractive regions provide different refractive focusing powers. The central zone has an adjustment in step height and/or curvature. By way of example, as shown schematically in FIG. 9, a central refractive region 90a of such an IOL 90 can contribute to the IOL's far-focus optical power (corresponding to far focus A) while an outer refractive region 90b of the IOL contributes—refractively—to the IOL's near-focus optical power (corresponding to near focus B). A diffractive region 90c, in turn, contributes—diffractively—to both the near and the far focusing powers of the IOL. Such a difference in the refractive focusing properties of the central and outer regions can be achieved, e.g., by configuring the outer region of one or both of the lens surfaces to have a different surface curvature (surface profile) than that of the respective central region.

In some cases, the base profile of at least one of the lens surfaces can exhibit a selected degree of asphericity to control aberrations, such as to control depth-of-focus. For example, the anterior surface on which a diffractive region is disposed can exhibit a spherical profile while the posterior surface exhibits a certain degree of asphericity. By way of example, further teachings regarding configuring one or more of the lens surfaces to have aspherical profiles can be found in pending U.S. patent application entitled “Intraocular Lens” having a Ser. No. 11/397,332, filed on Apr. 4, 2006, which is herein incorporated by reference.

In other cases, at least one of the lens surfaces can have a toric base profile (a profile characterized by two different curvatures along two orthogonal directions of the surface) to help correct astigmatism.

In some embodiments, the biocompatible polymeric material of the optic can be impregnated one or more dyes such that the lens can provide some degree of filtering of blue light. Some examples of such dyes are provided in U.S. Pat. Nos. 5,528,322 (entitled “Polymerizable Yellow Dyes And Their Use In Ophthalmic Lenses”), 5,470,932 (entitled “Polymerizable Yellow Dyes And Their Use In Ophthalmic Lenses”), 5,543,504 (entitled “Polymerizable Yellow Dyes And Their Use In Ophthalmic Lenses), and 5,662,707 (entitled “Polymerizable Yellow Dyes And Their Use In Ophthalmic Lenses), all of which are herein incorporated by reference.

A variety of known manufacturing techniques can be employed to form an ophthalmic lens (e.g., an IOL) in accordance with the teachings of the invention. For example, such techniques can be employed to initially form a refractive optic and subsequently generate an annular diffractive region on one of the surfaces of the optic such that the diffractive region would surround a central refractive region of the surface.

Those having ordinary skill in the art will appreciate the certain modifications can be made to the above embodiments without departing from the scope of the invention.

What is claimed is:

1. An intraocular lens (IOL), comprising
an optic having an anterior surface and a posterior surface, said optic having a central refractive region for providing one refractive focusing power, and
a diffractive region disposed on one of said surfaces so as to provide a near and a far diffractive focusing powers, wherein the diffractive region comprises a plurality of diffractive zones separated from one another by a plurality of steps and wherein the first step differs in height from the second step so as to alter the phase of the central refractive region to provide increased light to the refractive focusing power.

2. The IOL of claim 1, wherein each of said anterior and posterior surfaces includes a central refractive region.

3. The IOL of claim 2, wherein said central refractive region of any of the anterior and the posterior surface has a diameter in a range of about 0.5 mm to about 2 mm.

4. The IOL of claim 2, wherein said central refractive region of each of said anterior and the posterior surface has a substantially spherical profile.

5. The IOL of claim 4, wherein said diffractive region outside of said central refractive region has a substantially aspheric base profile.

6. The IOL of claim 2, wherein the diffractive region at least partially surrounds the central refractive region of the surface on which it is disposed.

7. The IOL of claim 1, wherein said far focusing power of the diffractive region corresponds substantially to said refractive focusing power provided by the optic's central refractive region.

8. The IOL of claim 1, wherein said optic comprises an outer refractive region.

9. The IOL of claim 8, wherein said outer refractive region provides a focusing power substantially equal to the refractive focusing power provided by the central region.

10. The IOL of claim 1, wherein at least one of said surfaces exhibits an aspheric base profile adapted to control aberrations of the lens.

11. The IOL of claim 1, wherein said central refractive region has a substantially spherical profile.

12. The IOL of claim 11, wherein said diffractive region outside of said central refractive region has a substantially aspheric base profile.

13. A method of correcting vision, comprising
providing an optic having an anterior surface and a posterior surface, said optic having:
a central refractive region for providing one refractive focusing power, and
a diffractive region disposed on one of said surfaces so as to provide a near and a far diffractive focusing powers, wherein the diffractive region comprises a plurality of diffractive zones separated from one another by a plurality of steps and wherein the first step differs in height from the second step so as to alter the phase of the central refractive region to provide increased light to the refractive focusing power; and
implanting said optic in a patient's eye.

14. A method of manufacturing an ophthalmic lens, comprising
forming an optic having an anterior surface and a posterior surface having base profiles adapted for generating a far-focus; and
generating a diffractive structure on at least one of said surfaces such that said surface comprises a central refractive region and an outer refractive region, said diffractive structure contributing to said far-focus optical power while also providing a near-focus optical power, wherein the diffractive structure comprises a plurality of diffractive zones separated from one another by a plurality of steps and wherein the first step height differs form that of the second step so as to alter the phase of the central refractive region to provide increased light to the far-focus optical power.
